(19) United States
(12) Patent Application Publication
(10) Pub. No.: US 2011/0041875 A1
(43) Pub. Date: Feb. 24, 2011

(54) CLEANING DEVICE AND CLEANING METHOD FOR MEDICINE EJECTING DEVICE

(75) Inventor: Yoshimasa Araki, Yokohama-shi (JP)

Correspondence Address: FITZPATRICK CELLA HARPER & SCINTO, 1290 Avenue of the Americas, NEW YORK, NY, 10104-3800, US

(73) Assignee: CANON KABUSHIKI KAISHA, Tokyo (JP)

(21) Appl. No.: 12/990,515

(22) PCT Filed: Jul. 3, 2009
(86) PCT No.: PCT/JP2009/062560

§ 371 Date: Nov. 1, 2010

(30) Foreign Application Priority Data
Jul. 4, 2008 (JP) 2008-175334

(51) Int. Cl. B08B 3/00

(52) U.S. Cl. 134/10; 134/184; 222/148

(57) ABSTRACT

A medicine ejecting device 1 having a medicine ejecting section 2 is mounted to a cleaning device 12. Then, the cap section 16 and the cleaning liquid feeding section 17 of the cleaning device 12 are connected respectively to the medicine ejecting section 2 and the cleaning liquid injecting section 5 of the medicine ejecting device 1. Cleaning liquid is supplied from the cleaning liquid storing section 13 of the cleaning device 12 to the cleaning liquid injecting section 5 and introduced into the medicine ejecting section 2 by way of a three-way changeover valve 8 by means of a cleaning liquid feeding pump 19. After the cleaning operation, the waste liquid is collected from the medicine ejecting section 2 to the waste liquid collecting section 14 by way of the cap section 16 of the cleaning device 12. Thus, the medicine ejecting device can be made compact and lightweight as a cleaning device storing cleaning liquid for cleaning the medicine ejecting section of the medicine ejecting device is arranged outside the medicine ejecting device.

### TECHNICAL FIELD

This invention relates to a cleaning device and a cleaning method for cleaning the medicine ejecting section of an inhalation type medicine ejecting device (inhaler) to be used by a user to inhale a medicine.

### BACKGROUND ART

There has been an increasing demand in recent years for means of administering a dose of medicine to the windpipe and the lungs of a user, or a patient, by ejecting the medicine as droplets and also for handy methods of causing a dose of medicine to be absorbed by way of the lungs of the user instead of injection so as to facilitate the medication by the user, or the patient, him or herself. Inhalers for generating bubbles in liquid medicine by means of a heater element arranged in an ejection head, causing medicine-containing liquid and hence the medicine to be ejected under the pressure of the generated bubbles into the air flow to be inhaled as droplets from the mouth of the user has been developed recently (see International Publications Nos. WO1995/001137 and WO2002/004043). They are inhalers designed to eject medicine on the basis of the so-called principle of thermal inkjet ejection.

The medicine ejecting device is provided with an ejection head that ejects medicine. The ejection head can be integrated with a medicine tank that contains medicine to operate as a medicine cartridge. In the case where the ejection head is used more than once, an appropriate means is required in order to ensure good hygiene of the ejection head.

Apart from medicine ejecting devices, inkjet recording apparatus require the ejection head thereof to be periodically cleaned because ink can be coagulated in the nozzle part and the ink flow path of the ejection head. For instance, Japanese Patent Application Laid-Open No. 2007-268997 discloses an ink ejecting apparatus wherein a pump and a changeover valve are linked to the ejection head of the apparatus by way of a tube. The changeover valve is linked to a cleaning liquid cartridge and also to an ink cartridge. To clean the ejection head, the cleaning liquid cartridge is made to communicate with the ejection head by operating the changeover valve. Then, cleaning liquid is supplied to the ejection head from the cleaning liquid cartridge to clean the ejection head as the pump is driven to operate. On the other hand, to print on a sheet of printing paper by means of the ejection head and ink, the ink cartridge is made to communicate with the ejection head by operating the changeover valve and then ink is supplied to the ejection head from the ink cartridge.

Now, to clean the medicine ejecting section of a medicine ejecting device disclosed in Japanese Patent Application Laid-Open No. 2007-268997, the medicine ejecting device needs to be provided with a cleaning liquid storing section. On the other hand, a medicine ejecting device such as an inhaler is normally borne by the user and hence is required to be compact and easily portable. A medicine ejecting device provided both with a cleaning liquid storing section and a medicine storing section will inevitably be bulky and heavy to a great inconvenience of the user.

### DISCLOSURE OF THE INVENTION

Therefore, the object of the present invention is to provide a cleaning device and a cleaning method for cleaning a medicine ejecting device that do not require the medicine ejecting device to be equipped with a cleaning liquid storing section.

According to the present invention, the above object is achieved by providing a cleaning device for cleaning a medicine ejecting section of a medicine ejecting device, including: a cleaning liquid collecting section that is separably connectable to a medicine ejecting side of the medicine ejecting section of the medicine ejecting device; a cleaning liquid feeding section that is separably connectable to a medicine feeding side of the medicine ejecting section of the medicine ejecting device; a cleaning liquid storing section that stores cleaning liquid for cleaning the medicine ejecting section of the medicine ejecting device; and a cleaning liquid feeding pump that delivers cleaning liquid from the cleaning liquid storing section to the cleaning liquid feeding section.

According to the present invention, there is also provided a medicine ejecting device including: a medicine ejecting section; a medicine feeding section that feeds medicine to the medicine ejecting section; a communication section that makes the medicine feeding section communicate with the medicine ejecting section; a flow path switching section arranged at the communication section; and a cleaning liquid injecting section communicating with the flow path switching section to cause the medicine ejecting device to receive cleaning liquid fed from a cleaning device, the cleaning device being independent from the medicine ejecting device, the flow path switching section being adapted to feed the cleaning liquid delivered to the cleaning liquid injecting section to the medicine ejecting section so as to clean the medicine ejecting section.

According to the present invention, there is also provided a cleaning method for cleaning a medicine ejecting section of a medicine ejecting device, including: a step of separably connecting the medicine ejecting device to a cleaning device storing cleaning liquid in order to clean the medicine ejecting section of the medicine ejecting device; a step of making the medicine ejecting section of the medicine ejecting device communicate with a cleaning liquid feeding section of the cleaning device; a step of cleaning the medicine ejecting section of the medicine ejecting device with the cleaning liquid fed from the cleaning device; a step of collecting the cleaning liquid to the cleaning device after cleaning the medicine ejecting section of the medicine ejecting device; and a step of separating the cleaned medicine ejecting device from the cleaning device.

Thus, according to the present invention, a cleaning device having a cleaning liquid storing section and other components is connected to the medicine ejecting section of a medicine ejecting device only at the time of a cleaning operation so that the medicine ejecting device is not required to have a cleaning liquid storing section therein. Then, as a result, the medicine ejecting device can be made compact and lightweight.

Other features and advantages of the present invention will be apparent from the following description taken in conjunction with the accompanying drawings, in which like reference characters designate the same or similar parts throughout the figures thereof.

### BRIEF DESCRIPTION OF THE DRAWINGS

FIG. 1 is a schematic front view of the medicine ejecting device of Example 1, illustrating an external appearance thereof.

FIG. 2 is a schematic lateral view of the medicine ejecting device of Example 1, illustrating an appearance thereof.

FIG. 3 is a schematic view of the medicine ejecting device of Example 1, illustrating the internal configuration thereof.

FIG. 4 is a schematic plan view of the cleaning device of Example 1, illustrating an external appearance thereof.

FIG. 5 is a schematic lateral view of the cleaning device of Example 1, illustrating an external appearance thereof.

FIG. 6 is a schematic view of the cleaning device of Example 1, illustrating the internal configuration thereof.

FIG. 7 is a schematic view of the medicine ejecting device of Example 1 fitted to the cleaning device, illustrating the internal configurations thereof.

FIG. 8 is a schematic view of the medicine ejecting device of Example 2 fitted to the cleaning device, illustrating the internal configurations thereof.

### BEST MODE FOR CARRYING OUT THE INVENTION

Preferred embodiments of the present invention will now be described in detail in accordance with the accompanying drawings.

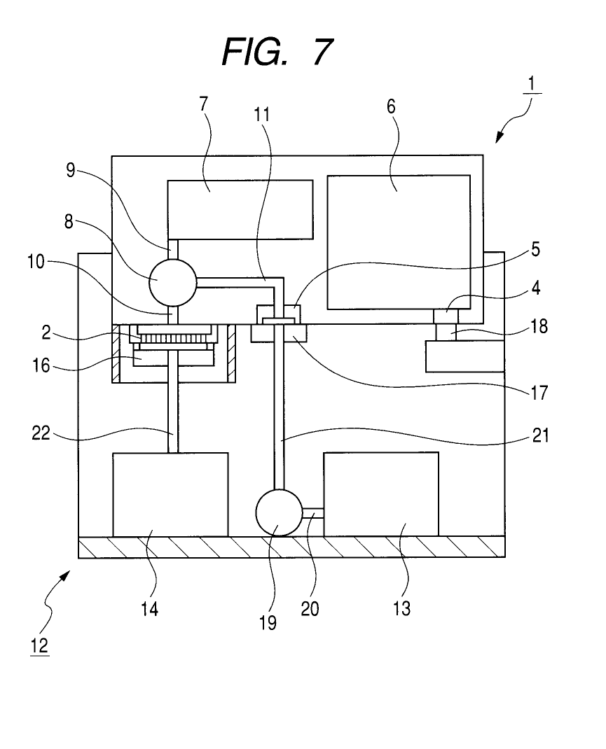

As illustrated in FIG. 7, the medicine ejecting device 1 has a medicine ejecting section 2 and a medicine cartridge 7 that operates as a medicine feeding section. The cleaning device 12 for cleaning the medicine ejecting section 2 of the medicine ejecting device 1 by turn has a cleaning liquid storing section 13 that stores cleaning liquid and a waste liquid storing section 14 that also operates as a cleaning liquid collecting section and stores the waste liquid (cleaning liquid) collected from the medicine ejecting side of the medicine ejecting section 2. Additionally, the cleaning device 12 has a cleaning liquid feeding section 17 that communicates with the cleaning liquid storing section 13 and a cleaning liquid feeding pump 19 connected to the cleaning liquid storing section 13. Thus, cleaning liquid is supplied from the cleaning device 12 to the medicine feeding side of the medicine ejecting section 2 of the medicine ejecting device 1 by means of the cleaning liquid feeding pump 19 to clean the medicine ejecting section 2.

The medicine ejecting section 2 is formed by an ejection head having a plurality of ejection ports. One or more than one elements for generating energy necessary for ejecting medicine are arranged corresponding to the plurality of ejection ports on a one to one basis, on a one to many basis or on a many to one basis. For instance, one or more than one electrothermal transducers such as micro-heaters are arranged in the case of ejecting medicine by applying heat to the medicine, utilizing the principle of thermal inkjet ejection. The ejection head may alternatively be a piezoelectric inkjet type ejection head that employs one or more than one piezoelectric elements as so many ejection energy generating elements. An inkjet type ejection head of any of the types under consideration has a large number of micro ejection ports (nozzles), which highly probably need to be periodically cleaned so as not to be clogged by medicine. For the purpose of the present invention, “the medicine ejecting side of the medicine ejecting section 2” refers to the side thereof exposed to the atmosphere relative to the plane of the ejection ports (the nozzles) of the ejection head, whereas “the medicine feeding side of the medicine ejecting section 2” refers to the inside of the ejection head, or the inside of the medicine ejecting device main body.

### Example 1

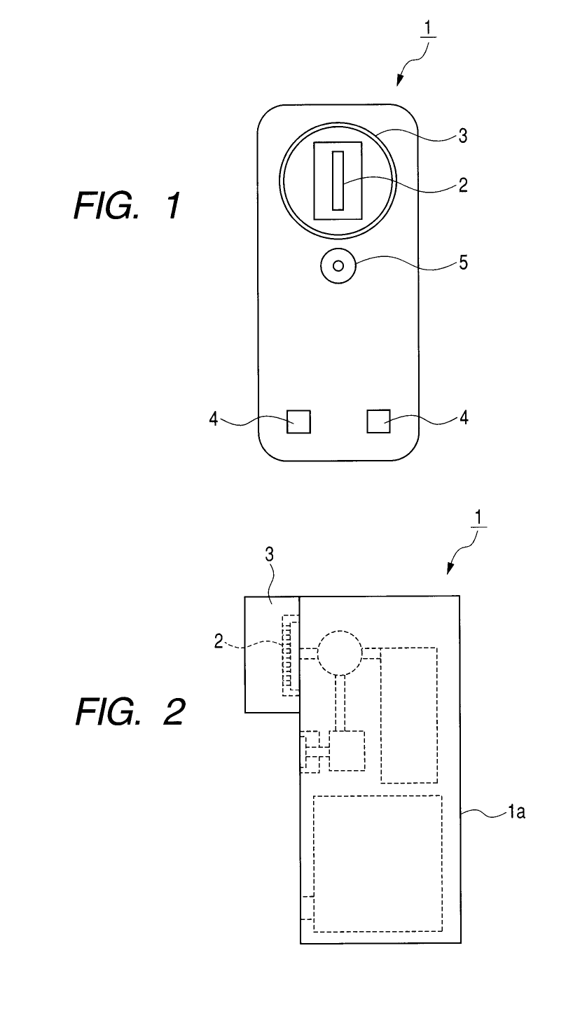

FIG. 1 is a schematic front view of the medicine ejecting device of Example 1, which is an inhalation type medicine ejecting device (inhaler), illustrating an external appearance thereof.

A medicine ejecting section 2 (ejection head) is arranged at the medicine ejecting device 1 and medicine is supplied from a medicine cartridge mounted in the medicine ejecting device 1 to the medicine ejecting section 2 and ejected from the latter. The medicine ejecting device 1 is provided with a mouthpiece 3. The user puts the mouthpiece 3 into his or her mouth and inhales medicine. The mouthpiece 3 can be cleaned if it is removable. The mouthpiece 3 can be disposed and replaced by a new one when it has been worn or broken. A customized mouthpiece may be employed to meet the requirements on the part of the user in terms of taste and convenience. The medicine ejecting device 1 is provided with a charging terminal 4 for charging the battery arranged in the medicine ejecting device 1. Medicine is ejected from the medicine ejecting section 2 as micro droplets of liquid medicine by an inkjet system that utilizes a thermal effect or a piezoelectric effect. A battery is employed as drive source for driving the medicine ejecting section 2 to eject medicine. The medicine ejecting device 1 is provided with a cleaning liquid injecting section 5. Thus, cleaning liquid is supplied from the cleaning liquid injecting section 5 to clean the medicine ejecting section 2.

FIG. 2 is a schematic lateral view of the medicine ejecting device 1, illustrating an appearance thereof. The user holds the medicine ejecting device main body 1a by hand and inhales medicine.

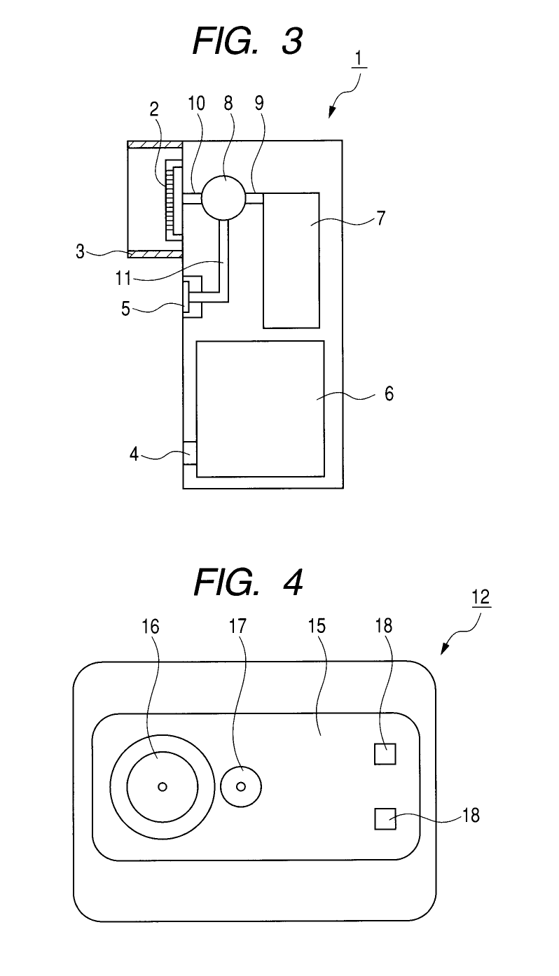

FIG. 3 is a schematic view of the medicine ejecting device 1, illustrating the internal configuration thereof. The medicine cartridge 7 is filled with the medicine injected thereinto so as to be ejected from the medicine ejecting section 2. The medicine cartridge 7 communicates with a three-way changeover valve 8 that is a flow path switching section by way of a first communication section 9 in an airtight manner. The three-way changeover valve 8 may be formed by combining two-way valves. The three-way changeover valve 8 also communicates with the medicine ejecting section 2 in an airtight manner by way of a second communication section 10. The three-way changeover valve 8 further communicates with the cleaning liquid injecting section 5 in an airtight manner by way of a third communication section 11. The cleaning liquid injecting section 5 is made of a flexible and airtight material. The cleaning liquid injecting section 5 is part of the medicine ejecting device 1 arranged on the surface of the cabinet of the latter and adapted to come into engagement with the cleaning liquid feeding section 17 of the cleaning device 12 when the cleaning device is mounted on the medicine ejecting device according to the present invention. In other words, the cleaning liquid injecting section 5 receives the cleaning liquid fed from the cleaning device that is independent from the medicine ejecting device. Specific examples of the material of the cleaning liquid injecting section 5 include rubber and other elastomers. The second communication section 10 is a flow path for feeding medicine or cleaning liquid to the ejection head 2. Both the first communication section 9 and the third communication section 11 are also flow paths.

To eject medicine from the medicine ejecting section 2, the three-way changeover valve 8 is so operated as to make the medicine cartridge 7 communicate with the medicine ejecting section 2. Under this condition, the cleaning liquid injecting section 5 does not communicate with the medicine ejecting section 2. To feed cleaning liquid from the cleaning liquid injecting section 5 to the medicine ejecting section 2 in order to clean the medicine ejecting section 2, the three-way changeover valve 8 is so switched as to make the cleaning liquid injecting section 5 communicate with the medicine feeding side of the medicine ejecting section 2. Under this condition, the medicine cartridge 7 does not communicate with the medicine ejecting section 2 and the charging terminal 4 is connected to the battery 6.

FIG. 4 is a schematic plan view of the cleaning device 12, illustrating an external appearance thereof. The cleaning device 12 has a medicine ejecting device mounting section 15 for mounting a medicine ejecting device 1 thereon. The cleaning device 12 has a cap section 16 that can be separably connected to the medicine ejecting section 2 of the medicine ejecting device 1 so as to be held to and communicate with the medicine ejecting side of the medicine ejecting section 2 in an airtight manner. The cap section 16 covers the plane of the ejection ports of the medicine ejecting section 2 so as to hermetically close and protect them. Furthermore, the cleaning device 12 has a cleaning liquid feeding section 17 that can be separably connected to the cleaning liquid injecting section 5 of the medicine ejecting device 1 so as to be held to and communicate with the cleaning liquid injecting section 5 in an airtight manner, and a contact 18 to be held in contact with the charging terminal 4 of the medicine ejecting device 1 in order to charge the battery 6.

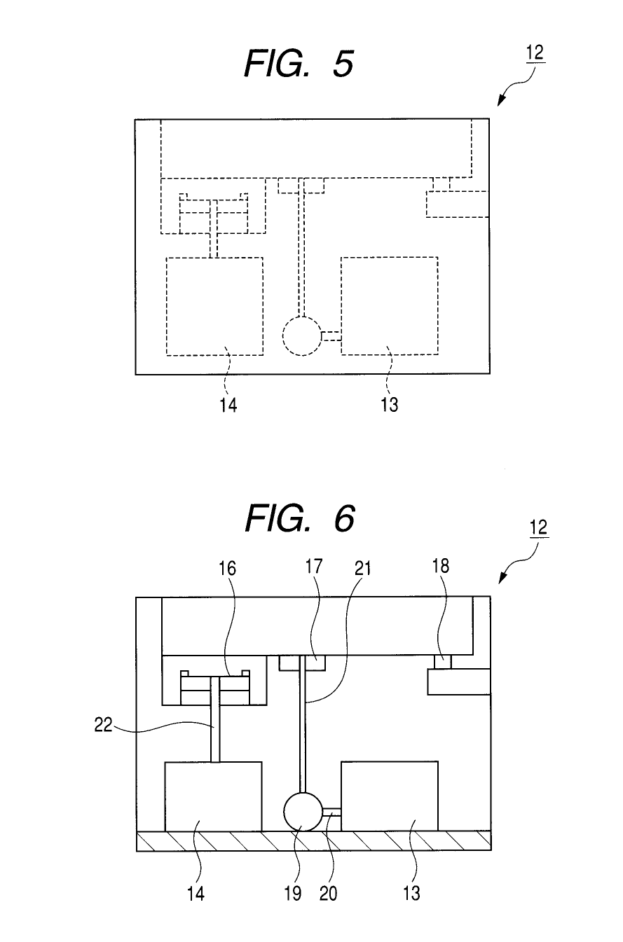

FIG. 5 is a schematic lateral view of the cleaning device 12, illustrating an external appearance thereof. A cleaning liquid storing section 13 and a waste liquid storing section 14 are arranged in the inside of the cleaning device 12.

FIG. 6 is a schematic view of the cleaning device 12, illustrating the internal configuration thereof. The cleaning liquid storing section 13 communicates with the cleaning liquid feeding pump 19 in an airtight manner by way of a fourth communication section (flow path) 20. The cleaning liquid feeding pump 19 communicates with the cleaning liquid feeding section 17 in an airtight manner by way of a fifth communication section (flow path) 21. The cap section 16 communicates with the waste liquid storing section 14 in an airtight manner by way of a sixth communication section (flow path) 22. The cap section 16 is formed using a flexible and airtight member. Specific materials that can be used for the cap section 16 include rubber and other elastomers. The contact 18 is connected to an electric circuit board (not shown).

FIG. 7 is a schematic view of the medicine ejecting device 1 fitted to the cleaning device 12, illustrating the internal configurations thereof. The medicine ejecting device 1 is fitted to the cleaning device 12 to clean the medicine ejecting section 2 of the medicine ejecting device 1.

The cleaning liquid injecting section 5 of the medicine ejecting device 1 is held to and communicates with the cleaning liquid feeding section 17 of the cleaning device 12 in an airtight manner, while the medicine ejecting section 2 of the medicine ejecting device 1 is held to and communicates with the cap section 16 of the cleaning device 12 in an airtight manner. The charging terminal 4 of the medicine ejecting device 1 is held in contact with the contact 18 and hence in a state of being able to electrically energize the battery.

To clean the medicine ejecting section 2, the three-way changeover valve 8 is so operated as to make the cleaning liquid injecting section 5 communicate with the medicine ejecting section 2. Then, cleaning liquid is supplied from the cleaning liquid storing section 13 to the cleaning liquid feeding pump 19 by way of the fourth communication section 20 and then to the cleaning liquid feeding section 17 by way of the fifth communication section 21 as the cleaning liquid feeding pump 19 is driven to operate. The cleaning liquid that is fed to the cleaning liquid feeding section 17 is then sent from the cleaning liquid injecting section 5 to the three-way changeover valve 8 by way of the third communication section 11. The cleaning liquid that is sent to the three-way changeover valve 8 is then supplied to the medicine ejecting section 2 by way of the second communication section 10. Then, the medicine ejecting section 2 is cleaned by the cleaning liquid. The cleaning liquid that is used for the cleaning operation (waste liquid) is then collected from the cap section 16 to the waste liquid storing section 14 by way of the sixth communication section 22.

After the operation of cleaning the medicine ejecting section 2, the three-way changeover valve 8 is operated so as to make the medicine cartridge 7 communicate with the medicine ejecting section 2. The cleaning liquid remaining in the medicine ejecting section 2, the second communication section 10 and the three-way changeover valve 8 is ejected out of the medicine ejecting section 2 as the medicine ejecting section 2 is operated for ejection. Then, medicine is supplied from the medicine cartridge 7 to the medicine ejecting section 2 by way of the first communication section 9, the three-way changeover valve 8 and the second communication section 10 and ejected from the medicine ejecting section 2. In this way, the cleaning liquid in the medicine ejecting section 2, the second communication section 10 and the three-way changeover valve 8 can be replaced by medicine.

### Example 2

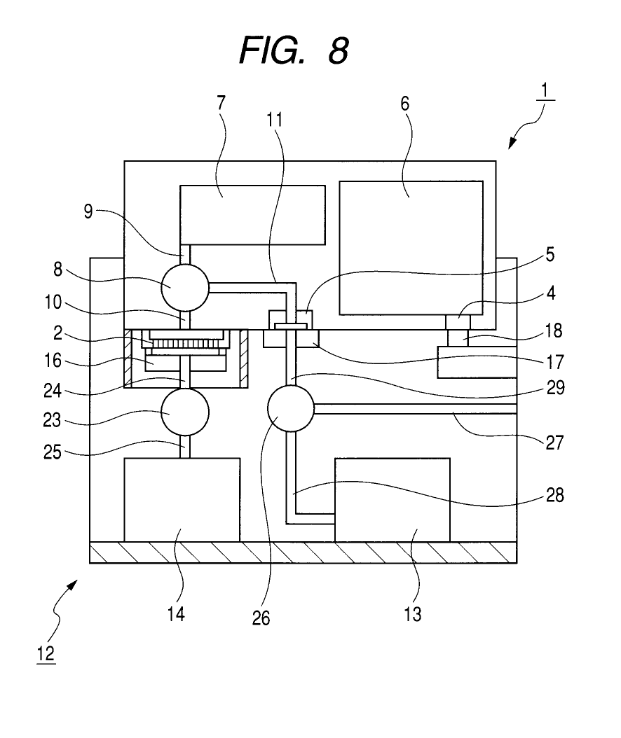

FIG. 8 is a schematic view of the cleaning device fitted to the medicine ejecting device of Example 2, illustrating the internal configurations thereof. In this example, the cleaning liquid feeding pump 19 of Example 1 is replaced by a cleaning liquid collecting pump 23 that operates like the cleaning liquid feeding pump 19 and connected between the cap section 16 and the waste liquid storing section 14 of the cleaning device 12. Additionally, an open-to-air three-way changeover valve 26 is arranged at the connecting section of the cleaning liquid storing section 13 and the cleaning liquid feeding section 17 and provided with an air vent section 27. Otherwise, the cleaning device of this example is same as and identical with that of Example 1 and hence will not be described any further.

As illustrates in FIG. 8, the cleaning liquid collecting pump 23 communicates with a seventh communication section 24 and also with an eighth communication section 25 both in an airtight manner. The seventh communication section 24 communicates with the cap section 16 in an airtight manner, while the eighth communication section 25 communicates with the waste liquid storing section 14 in an airtight manner. The open-to-air three-way changeover valve 26 communicates with a ninth communication section 28 and a tenth communication section 29 and also with the air vent section 27 in an airtight manner. The other end of the air vent section 27 is exposed to the atmosphere.

To clean the medicine ejecting section 2, the three-way changeover valve 8 at the side of the medicine ejecting device is so operated as to make the cleaning liquid injecting section 5 communicate with the medicine ejecting section 2. Additionally, the open-to-air three-way changeover valve 26 at the side of the cleaning device is operated so as to make the ninth communication section 28 and the tenth communication section 29 communicate with each other. Then, cleaning liquid is sent from the cleaning liquid storing section 13 to the cleaning liquid feeding section 17 by way of the ninth communication section 28, the open-to-air three-way changeover valve 26 and the tenth communication section 29 as the cleaning liquid collecting pump 23 is driven to operate. The cleaning liquid supplied to the cleaning liquid feeding section 17 is then sent from the cleaning liquid injecting section 5 to the three-way changeover valve 8 by way of the third communication section 11. The cleaning liquid supplied to the three-way changeover valve 8 is then fed to the medicine ejecting section 2 by way of the second communication section 10. Then, the medicine ejecting section 2 is cleaned by the cleaning liquid. The cleaning liquid that is used for the cleaning operation (waste liquid) is then sent from the cap section 16 to the cleaning liquid collecting pump 23 by way of the seventh communication section 24. The cleaning liquid that is used for the cleaning operation (waste liquid) is then discharged from the cleaning liquid collecting pump 23 into the waste liquid storing section 14 by way of the eighth communication section 25.

After the operation of cleaning the medicine ejecting section 2, the three-way changeover valve 8 is operated so as to make the medicine cartridge 7 communicate with the medicine ejecting section 2. Then, medicine can be supplied from the medicine cartridge 7 to the medicine ejecting section 2 to replace the cleaning liquid in the medicine ejecting section 2 by driving the cleaning liquid collecting pump 23 to operate.

Alternatively, after the operation of cleaning the medicine ejecting section 2 with cleaning liquid, the open-to-air three-way changeover valve 26 may be operated to make the air vent section 27 communicate with the ninth communication section 28 so as to suck air by way of the air vent section 27 and supply the air to the medicine ejecting section 2. Then, most of the cleaning liquid in the medicine ejecting section 2 can be discharged into the waste liquid storing section 14. As a result, the quantity of cleaning liquid remaining in the medicine ejecting section 2 can be remarkably reduced to by turn reduce the quantity of medicine injected into the medicine ejecting section 2 in order to replace the cleaning liquid in the medicine ejecting section 2. The open-to-air three-way changeover valve 26 may be formed by combining two-way valves. A filter may be arranged in the air vent section 27 in order to prevent dusts, dirt and liquid droplets floating in air from entering the air vent section 27 in the case where air is sucked through the air vent section 27.

The present invention is not limited to the above embodiments and various changes and modifications can be made within the spirit and scope of the present invention. Therefore, to apprise the public of the scope of the present invention, the following claims are made.

This application claims the benefit of Japanese Patent Application No. 2008-175334, filed Jul. 4, 2008, which is hereby incorporated by reference in its entirety.

1. A cleaning device for cleaning a medicine ejecting section of a medicine ejecting device, comprising:
a cleaning liquid collecting section that is separably connectable to a medicine ejecting side of the medicine ejecting section of the medicine ejecting device;
a cleaning liquid feeding section that is separably connectable to a medicine feeding side of the medicine ejecting section of the medicine ejecting device;
a cleaning liquid storing section that stores cleaning liquid for cleaning the medicine ejecting section of the medicine ejecting device; and
a cleaning liquid feeding pump that delivers cleaning liquid from the cleaning liquid storing section to the cleaning liquid feeding section.

2. The device according to claim 1, wherein the cleaning liquid collecting section has a cap section communicating with the medicine ejecting side of the medicine ejecting section of the medicine ejecting device and a waste liquid storing section communicating with the cap section.

3. The device according to claim 2, wherein the cleaning liquid feeding pump is replaced by a cleaning liquid collecting pump for collecting cleaning liquid to the waste liquid storing section by way of the cap section.

4. The device according to claim 3, wherein an air vent section is provided in the communication section for connecting the cleaning liquid storing section to the cleaning liquid feeding section.

5. A medicine ejecting device comprising:
a medicine ejecting section;
a medicine feeding section that feeds medicine to the medicine ejecting section;
a communication section that makes the medicine feeding section communicate with the medicine ejecting section;
a flow path switching section arranged at the communication section; and
a cleaning liquid injecting section communicating with the flow path switching section to cause the medicine ejecting device to receive cleaning liquid fed from a cleaning device, the cleaning device being independent from the medicine ejecting device,
the flow path switching section being adapted to feed the cleaning liquid delivered to the cleaning liquid injecting section to the medicine ejecting section so as to clean the medicine ejecting section.

6. A cleaning method for cleaning a medicine ejecting section of a medicine ejecting device, comprising:
a step of separably connecting the medicine ejecting device to a cleaning device storing cleaning liquid in order to clean the medicine ejecting section of the medicine ejecting device;
a step of making the medicine ejecting section of the medicine ejecting device communicate with a cleaning liquid feeding section of the cleaning device;
a step of cleaning the medicine ejecting section of the medicine ejecting device with the cleaning liquid fed from the cleaning device;
a step of collecting the cleaning liquid to the cleaning device after cleaning the medicine ejecting section of the medicine ejecting device; and
a step of separating the cleaned medicine ejecting device from the cleaning device.
